(19) United States
(12) Patent Application Publication
(10) Pub. No.: US 2024/0268751 A1
(43) Pub. Date: Aug. 15, 2024

(54) DEVICE, SYSTEM, AND METHOD FOR MONITORING WOUNDS

(71) Applicant: Edj Technology Inc., Brooklyn, NY (US)

(72) Inventors: DEEMA ABDEL MEGUID, Brooklyn, NY (US); ERICA KREISBERG, Brooklyn, NY (US); JUSTIN FREEMAN, Brooklyn, NY (US)

(73) Assignee: Edj Technology Inc., Brooklyn, NY (US)

(21) Appl. No.: 18/426,417

(22) Filed: Jan. 30, 2024

Related U.S. Application Data
(60) Provisional application No. 63/484,616, filed on Feb. 13, 2023.

(51) Int. Cl. A61B 5/00; A61B 5/01

(52) U.S. Cl. CPC A61B 5/445; A61B 5/0008; A61B 5/01; A61B 5/6833; A61B 5/7221; A61B 2560/0443; A61B 2562/0271; A61B 2562/043; A61B 2562/166

(57) ABSTRACT

An infection detection device is configured to continuously monitor a temperature of a periwound of a user and includes a dock and a drive. The dock includes an adhesive layer having an adhesive coating on a wound side thereof, a first dock portion and a second dock portion. The drive is configured to be inserted into a cavity of the dock and includes a printed circuit board, a plurality of first temperature sensors and at least one second temperature sensor. The printed circuit board is positioned along a base of the drive, and has a first printed circuit board portion and a second printed circuit board portion. The plurality of first temperature sensors extend from the first printed circuit board portion and through the first dock portion. The at least one second temperature sensor extends from the second printed circuit board portion and through the second dock portion.

### CROSS REFERENCE TO RELATED CO-PENDING APPLICATIONS

This application claims the benefit of U.S. provisional application Ser. No. 63/484,616 filed on Feb. 13, 2023 and entitled “Device, system, and method for monitoring wounds”, which is commonly assigned and the contents of which are expressly incorporated herein by reference.

### FIELD OF THE INVENTION

The present invention relates to a device, a system and a method for monitoring wounds, and in particular to a device that is applied near surgical sites of a patient for monitoring surgical site infections.

### BACKGROUND OF THE INVENTION

Each year in the United States alone, there are 44 million wounds, including chronic, non-healing, acute, or surgical wounds, among others. Complications from acute and chronic wounds combined add up to a net cost of approximately $30 billion dollars. Surgical Site Infections (SSI) are the most common and costly of all hospital-acquired infections. Each year in the United States, 20 million surgeries are performed, and 300,000 patients will develop an SSI. Readmission due to the occurrence of SSIs significantly burdens the healthcare system. Valuable resources such as hospital beds and operating rooms are then used for the treatment of advanced surgical site infections, adding up to a net cost of approximately seven billion dollars. There is a need to alleviate the burden of SSIs on both patients and the healthcare system by providing semi-autonomous wound care to patients.

Often, infection can be inevitable, meaning that despite preventative measures taken during the surgery and afterwards when caring for the wound, prevention is not always successful. While there are efforts in prevention and methods for treating SSIs, there is large deficit in diagnostics. There is a need for a diagnostic device that is separable into components for ease of dressing change, potential troubleshooting, and replacement if needed. There is also a need for the device to be compatible with a variety of wound dressings and to be capable of application without a dressing and to be as modular as possible.

### SUMMARY OF THE INVENTION

The present invention refers to a device, a system and a method for monitoring wounds, and in particular to a device that is applied near surgical sites of a patient for monitoring surgical site infections.

In general, in one aspect the invention provides an infection detection device configured to continuously monitor a temperature of a periwound of a user. The device includes a dock and a drive. The dock includes an adhesive layer having an adhesive coating on a wound side thereof, a first dock portion and a second dock portion. The drive is configured to be inserted into a cavity of the dock and includes a printed circuit board, a plurality of first temperature sensors and at least one second temperature sensor. The printed circuit board is positioned along a base of the drive, and has a first printed circuit board portion and a second printed circuit board portion. The plurality of first temperature sensors extend from the first printed circuit board portion and through the first dock portion. The at least one second temperature sensor extends from the second printed circuit board portion and through the second dock portion.

Implementations of this aspect of the invention include one or more of the following. The dock further includes a coupling mechanism configured to couple the drive to the dock. The coupling mechanism is configured to be inserted into a receiving element of the drive to couple the drive to the dock. The first dock portion includes a first opening extending through the adhesive layer to the periwound and the second dock portion includes a second opening extending through the adhesive layer to a portion of skin adjacent to the periwound. The first dock portion includes a first conductive layer spanning the first opening, the first conductive layer contacting the periwound and the first temperature sensors, and the second dock portion includes a second conductive layer spanning the second opening, the second conductive layer contacting the portion of skin adjacent to the periwound and the at least one second temperature sensor. The first dock portion includes a plurality of openings, each of the plurality of openings sized and shaped to receive a corresponding one of the first temperature sensors therethrough. Each of the plurality of openings including a conductive layer contacting the periwound and the corresponding one of the first temperature sensors. The dock is contoured to correspond to a shape of an incision. The drive is configured to be inserted into the cavity of the dock such that a top surface of the drive is flush with a top surface of the dock.

In general, in another aspect the invention provides a system for an infection detection system configured to continuously monitor a temperature of a periwound. The system includes an infection detection device, and a microcontroller. The infection detection device includes a dock and a drive. The dock is coupled to an adhesive layer having an adhesive coating on a wound side thereof, and has a measurement portion and a reference portion. The drive is configured to be inserted into a cavity of the dock and includes a printed circuit board, a plurality of first temperature sensors, and at least one second temperature sensor. The printed circuit board is positioned along a base of the drive, and has a first printed circuit board portion and a second printed circuit board portion. The plurality of first temperature sensors extend from the first printed circuit board portion and through the first dock portion. The at least one second temperature sensor extends from the second printed circuit board portion and through the second dock portion. The microcontroller is configured to compare measurement data from the plurality of first temperature sensors and reference data from the at least one second temperature sensor, and the infection detection device is configured to communicate with a remote communication device.

Implementations of this aspect of the invention include one or more of the following. The microcontroller is configured to transmit the comparison of the measurement data and the reference data and an output of the microcontroller to the remote communication device. The microcontroller is configured to transmit the measurement data and the reference data to the remote communication device. The dock further comprises a coupling mechanism configured to couple the drive to the dock. The first dock portion includes a first opening extending through the adhesive layer to the periwound and the second dock portion includes a second opening extending through the adhesive layer to a portion of skin adjacent to the periwound. The first dock portion includes a first conductive layer spanning the first opening, the first conductive layer contacting the periwound and the first temperature sensors, and the second dock portion includes a second conductive layer spanning the second opening, the second conductive layer contacting the portion of skin adjacent to the periwound and the at least one second temperature sensor. The first dock portion includes a plurality of openings, each of the plurality of openings sized and shaped to receive a corresponding one of the first temperature sensors therethrough. Each of the plurality of openings including a conductive layer contacting the periwound and the corresponding one of the first temperature sensors. The system further includes a bandage configured to cover the infection detection device and the periwound.

In general, in another aspect the invention provides a method for determining whether a wound in infected, including the following. First, collecting measurement data by measuring temperatures of periwound surrounding the wound via a plurality of first temperature sensors. Next, collecting reference data by measuring a temperature of a portion of skin adjacent to the periwound via at least one second temperature sensor. Next, filtering erroneous temperatures from the measurement data and the reference data. Next, comparing the filtered measurement data to the filtered reference data and then outputting an indication of a wound infection based on the comparison of the filtered measurement data to the filtered reference data. The plurality of first temperature sensors extend from a first portion of a printed circuit board of a drive, the at least one second temperature sensor extends from a second portion of the printed circuit board of the drive, and the drive is configured to be inserted into a cavity of a dock couplable to at least one of the periwound and the portion of skin adjacent to the periwound via an adhesive layer. The method further includes displaying the output on a remote communication device. The method further includes collecting and entering additional measurement data. The additional measurement data may be one or more of pain scale ratings, body temperature, ambient temperature, chills and sweat data, redness, soreness and swelling data, contact bleeding data, wound discharge data, wound odor data, vomiting, wound healing progress data, wound tissue granulation data, wound culture data, blood pressure data, heart rate data, oximeter data, level of consciousness, full blood count data, white blood cell count data, C-reactive protein data, plasma viscosity, erythrocyte sedimentation rate, microbial DNA data, and bacterial protease activity.

### BRIEF DESCRIPTION OF THE DRAWINGS

Referring to the figures, wherein like numerals represent like parts throughout the several views:

FIG. 1A depicts an infection detection system according to an exemplary embodiment of the present disclosure;

FIG. 1B depicts an infection detection system according to another exemplary embodiment of the present disclosure;

FIG. 2 depicts an exemplary user device of the infection detection system of FIG. 1;

FIG. 3 depicts a perspective view of a disassembled infection detection device in the system of FIG. 1;

FIG. 4 depicts a perspective view of the assembled infection detection device of FIG. 3;

FIG. 5 depicts a perspective view of a dock of the infection detection device of FIG. 3;

FIG. 5A depicts a perspective view of another embodiment of a disassembled infection detection device in the system of FIG. 1;

FIG. 5B depicts a perspective view of the assembled infection detection device of FIG. 5A;

FIG. 5C depicts a perspective view of another embodiment of the dock of the infection detection device of this invention;

FIG. 5D depicts a perspective view of another embodiment of a disassembled infection detection device in the system of FIG. 1;

FIG. 5E depicts a side perspective view of the assembled infection detection device of FIG. 5D;

FIG. 5F depicts a perspective view of the dock of the infection detection device of FIG. 5D mounted on the adhesive layer 110;

FIG. 5G depicts a perspective bottom view of the dock of the infection detection device of FIG. 5D and a perspective bottom view of the adhesive layer 110;

FIG. 5H depicts a perspective view of another embodiment of a disassembled infection detection device in the system of FIG. 1;

FIG. 5I depicts an enlarged perspective partial view of the disassembled infection detection device of FIG. 5H;

FIG. 5J depicts a perspective view of another embodiment of a disassembled infection detection device in the system of FIG. 1;

FIG. 6 depicts a top view of the dock of FIG. 5;

FIG. 7 depicts a side view of the infection detection device of FIG. 4;

FIG. 8 depicts a bottom perspective view of a drive of the infection detection device of FIG. 3;

FIG. 9 depicts an exploded perspective view of the drive of FIG. 8;

FIG. 10A shows a top view of a Printed Circuit Board (PCB) of the drive of FIG. 8;

FIG. 10B shows a bottom view of the PCB of FIG. 10A;

FIG. 11 is a schematic block diagram of a microcontroller of the drive of FIG. 8;

FIG. 12 depicts an exemplary embodiment of a flow diagram of a method for determining whether a wound is infected; and

FIG. 13 depicts another exemplary embodiment of a flow diagram for determining whether a wound is infected.

### DETAILED DESCRIPTION OF THE INVENTION

The present invention refers to a device, a system and a method for monitoring wounds, and in particular to a device that is applied near surgical sites of a patient for monitoring surgical site infections.

Exemplary embodiments of the present invention describe an infection detection device including a dock, a drive, a processor, and temperature sensors. Although the exemplary embodiments included herein describe infection detection devices that are used to monitor temperature and infection in and around surgical wounds (e.g., caesarean cuts and incisions), those skilled in the art will understand that the infection detection devices as described herein may be used in any application in which it is necessary to continuously measure a temperature on the body. Further, the infection detective device may be configured to detect infection in and around surgical wounds through temperature markers without contacting the surgical wounds and regardless of the bacteria infecting the surgical wounds. The infection detection device may be used for early detection of infection in and around surgical wounds and may be configured to conform to any surgical wound form. Moreover, the infection detection device may be configured to communicate with a remote user device (e.g., a computer, a tablet, a smartphone, etc.) for ease of access for the user. The remote user device may have a processor configured to run a software that interprets data received from the infection detection device and user inputs from the application to output an indication of a possible wound infection.

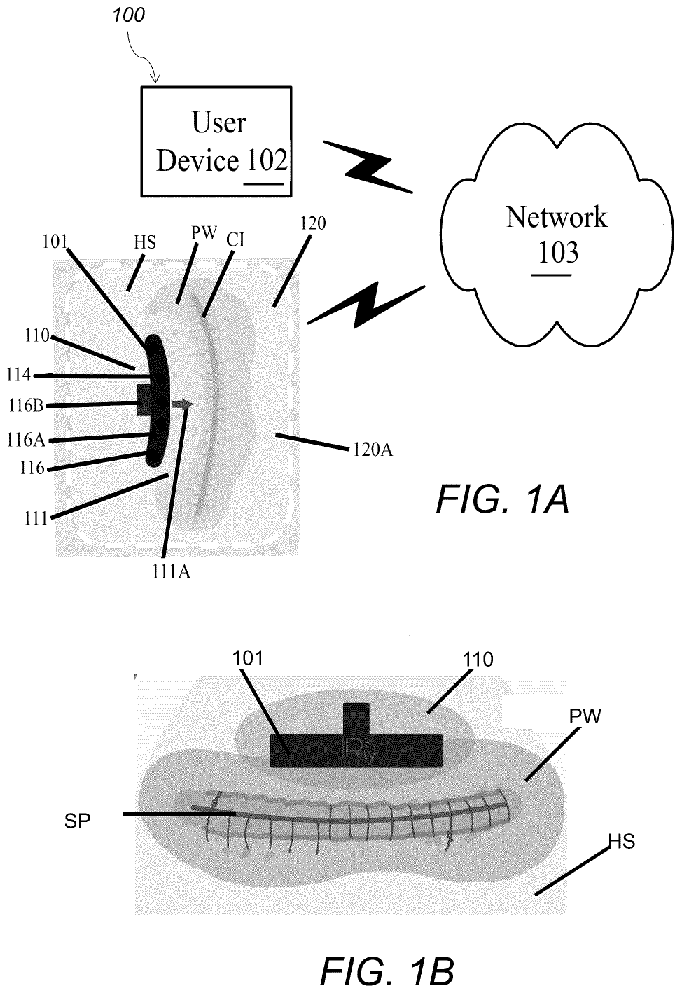

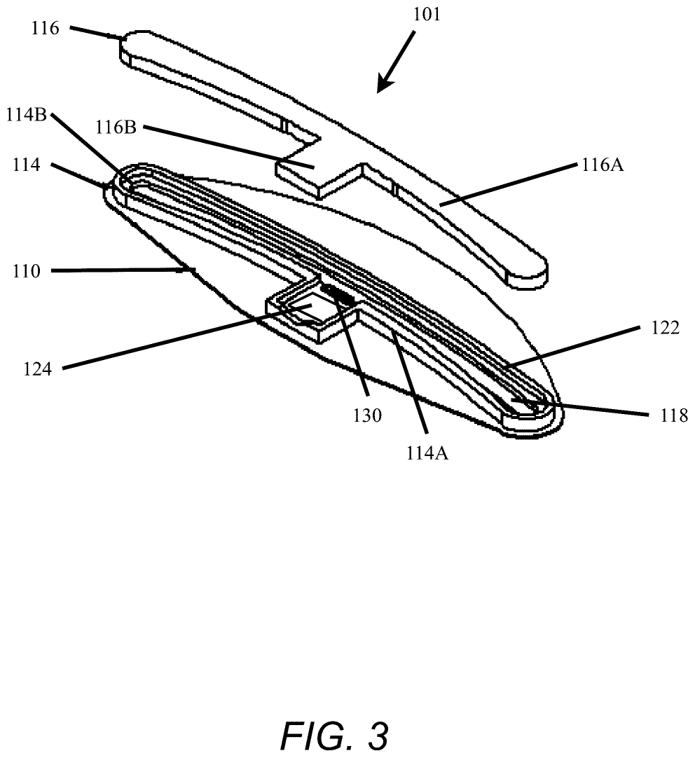

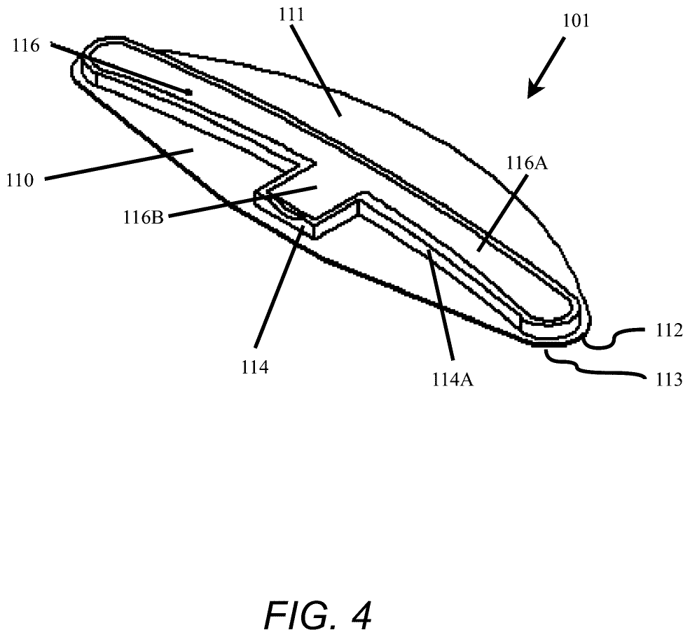

Referring to FIG. 1A, FIG. 1B, FIG. 3, and FIG. 4, an infection detection system 100 for monitoring a wound (e.g. caesarean incision CI, or any other type of skin perforation SP) of a user includes an infection detection device 101, a user device 102, a network 103, and a wound area 120A. The detection device 101 includes a drive 116 and a dock 114. The dock may be disposed on top of an adhesive layer 110 having a top surface 111 and an indicator 111A. The drive 116 includes a first monitor section 116A and a second monitor section 116B. A patient's wound area 120A includes a wound (shown as a caesarean incision CI or skin perforation SP), periwound area PW that is adjacent to the wound, and healthy skin HS. The periwound PW (i.e., periwound, peripheral wound area, wound bed) includes skin and tissue surrounding the wound that could become inflamed, become heated, and/or experience a temperature change, indicating that bacteria accessed the wound and the wound became infected. During operation of the infection detection device 101 (e.g., after a surgery), a user may place the infection detection device 101 on the periwound PW and the healthy skin HS sections so that the first monitor section 116A is disposed on top of at least a portion of the periwound PW section, and the second monitor section 116B is disposed on top of at least a portion of the healthy skin HS section. Data (e.g., temperature) of the periwound PW and the healthy skin HS sections are collected by the respective first monitor 116A and second monitor 116B sections, and transmitted to the user device 102 via network 103. A user, such as a nurse, doctor, any medical professional, or even the patient themselves, may use the user device 102 to determine if the wound is infected. In an embodiment, the infection detection device 101 may determine if the wound is infected. In an embodiment, the system 100 includes a bandage 120 configured to cover and secure the infection detection device 101, as shown in FIG. 1A. In another embodiment, the infection detection device 101 may adhere to the user via the adhesive layer 110, as shown in FIG. 1A and FIG. 1B.

In continued reference to FIG. 1A, FIG. 1B, FIG. 3 and FIG. 4, the top surface 111 of the adhesive layer 110 includes an indicator 111A that informs the user how to align the infection detection device 101 relative to the wound CI. The indicator 111A may be in the form of an arrow, as shown in FIG. 1A. When the infection detection device 101 is attached to the user's periwound PW and healthy skin HS sections, the top surface 111 faces away from the user's skin and the bottom surface 112, including the adhesive coating 113, faces toward the user's skin.

In continued reference to FIG. 1A, FIG. 1B, FIG. 3 and FIG. 4, the system 100 further includes the bandage 120. In one embodiment, the bandage 120 covers at least a portion of the infection detection device 101, the periwound PW and the wound section, i.e., caesarean incision CI or SP. The bandage 120 may be any bandage that is normally used for covering a wound or surgical incision, such as PRINEO®, Tegaderm™, AQUACEL®, a gauze cloth, a hydrogel, a film, a natural plaster, or a synthetic plaster, among others. However, in another exemplary embodiments, the system 100 does not require a bandage as the infection detection device 101 is configured to attach to the skin of the user via the adhesive coating 113 on the second surface 112 of the adhesive layer 110 of the infection detection device 101, as shown in FIG. 1B.

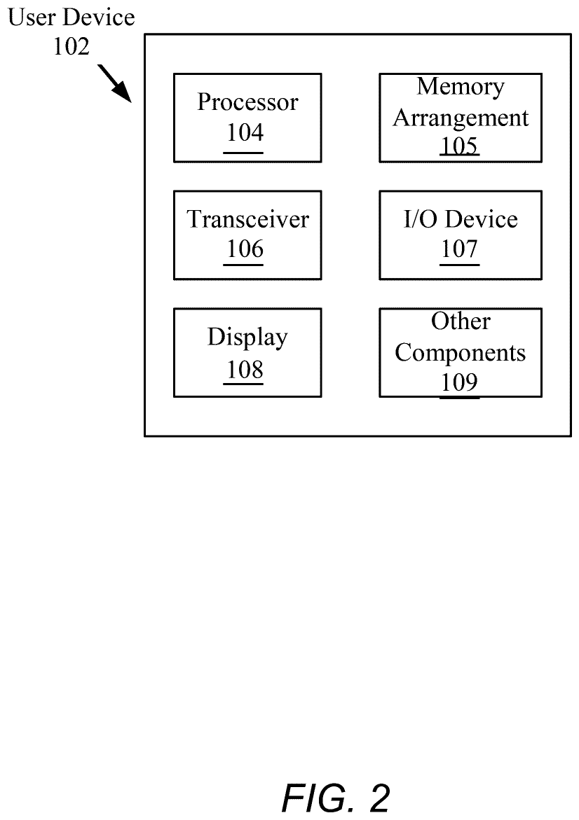

Referring to FIG. 2, the user device 102 includes a processor 104, a memory arrangement 105, a transceiver 106, an input/output (I/O) device 107, a display device 108, and other components 109. The processor 104 of this embodiment is configured to perform various procedures between the user device 102 and the network 103 (shown in FIG. 1A). In the exemplary embodiment, the processor 104 executes a program that interprets the collected data from the infection detection device 101 to make a determination about whether the caesarean incision CI is infected. In one example, the processor 104 of this embodiment determines, based on the collected data from the infection detection device 101, that the caesarean incision CI is infected and the user needs to urgently visit a doctor or physician to treat the infection. In further exemplary embodiments, the processor 104 is split among two or more processors to execute the program or a plurality of programs. For example, in one of the further exemplary embodiments a first processor interprets a first set of the collected data and a second processor interprets a second set of the collected data to determine whether the caesarean incision CI is infected.

In continued reference to FIG. 2, the memory arrangement 105 of this embodiment is a hardware component configured to store data related to operations performed by the user device 102. In the exemplary embodiment, the memory arrangement 105 is configured to store the collected data from the infection detection device 101 (shown in FIG. 1A, and FIG. 1B), as well as data output by the processor 104. The transceiver 106 in this embodiment is a hardware component configured to establish a connection with the network 103 and the infection detection device 101 to receive the collected data. Accordingly, the transceiver 106 operates on a variety of different frequencies. The input/output (I/O) device 107 is a hardware component that enables a user (e.g., a user, doctor, a nurse, a physician) to enter inputs. The display device 108 is a hardware component configured to convey to the user whether the caesarean incision CI is infected based on the collected data. The display device 108 also conveys to the user whether a portion of the infection detection device 101 needs to be changed. The other components 109 include, an audio input device, an audio output device, a power supply, a data acquisition device, ports to electrically connect the user device 102 to the infection detection device 101, among others. In an exemplary embodiment, the user device 102 is the user's cellphone. In a further exemplary embodiment, the user device 102 has an application configured to communicate with the infection detection device 101 (shown in FIG. 1A).

Referring to FIG. 3, the infection detection device 101 in a disassembled configuration includes the drive 116 detached from the dock 114. The drive 116 includes the first monitor section 116A and the second monitor section 116B. The dock 114 includes an exterior wall 114A forming a cavity 118 and a step 114B extending inward from the exterior wall 114A. A user inserts the drive 116 into the cavity 118, where the drive 116 will rest on the step 114B. The exterior wall 114A of the dock 114 substantially surrounds the drive 116, including both the first and second monitor sections 116A and 116B, as shown in FIG. 4. The exterior wall 114A forms a first dock section 122 and a second dock section 124, such that, when the drive 116 is inserted into the cavity 118, the first monitor section 116A of the drive 116 is disposed within the first dock section 122 and the second monitor section 116B is disposed within the second dock section 124.

In continued reference to FIG. 3, a dock coupling mechanism 130 is disposed on the dock 114 in between the first dock section 122 and the second dock section 124, where the dock coupling mechanism 130 connects the dock 114 to the drive 116. In an embodiment, the dock coupling mechanism 130 connects to a drive coupling mechanism 132 (shown in FIG. 8) disposed in the drive 116. The dock coupling mechanism 130 effectively separates the dock 114 into the first dock section 122 and the second dock section 124. In an embodiment, the infection detection device 101 does not include the adhesive layer 110.

Referring to FIG. 4, the infection detection device 101 in an assembled configuration includes the drive 116 inserted into cavity 118 of the dock 114. Also shown in FIG. 4 is the adhesive layer 110 having a top surface 111 and a bottom surface 112. In the embodiment, the dock 114 is coupled to the top surface 111 of the adhesive layer 110. The bottom surface 112 is configured to be flush with the skin of a patient and includes an adhesive coating 113 that is used to attach the infection detection device 101 onto the skin of the patient. In an embodiment, the adhesive coating 113 covers a portion of the bottom surface 112. In an embodiment, the adhesive coating 113 covers the entirety of the bottom surface 112. During operation of the infection detection device 101 (e.g., after a surgery), the infection detection device 101 is attached to the periwound PW (shown in FIG. 1A and FIG. 1B) of a user by placing the adhesive coated bottom surface 112 onto the periwound PW. The adhesive coating 113 may be any medical grade adhesive capable of attaching the dock 114 to the periwound PW of the user's skin. For example, the adhesive coating 113 may be an adhesive hydrogel (e.g., acrylic adhesives, pressure sensitive adhesives). In an exemplary embodiment, the adhesive layer 110 may detach from the dock 114.

In continued reference to FIG. 4, in a further exemplary embodiment, the adhesive layer 110, along with the dock 114 and the drive 116, are detached from the skin of a user. After the adhesive layer 110 is detached, the drive 116 may be removed from the cavity 118 of the dock 114. Next, a new adhesive layer 110, along with a new dock 114, may be attached to the skin of the user to replace the old adhesive layer 110 and the old dock 114. The drive 116 (i.e., the old drive 116) may then be inserted into a new cavity 118 of the new dock 114 in a substantially similar manner as the drive 116 was inserted into the old cavity 118 of the old dock 114. Accordingly, the adhesive layer 110 and the dock 114 may be replaced numerous times without replacing the drive 116.

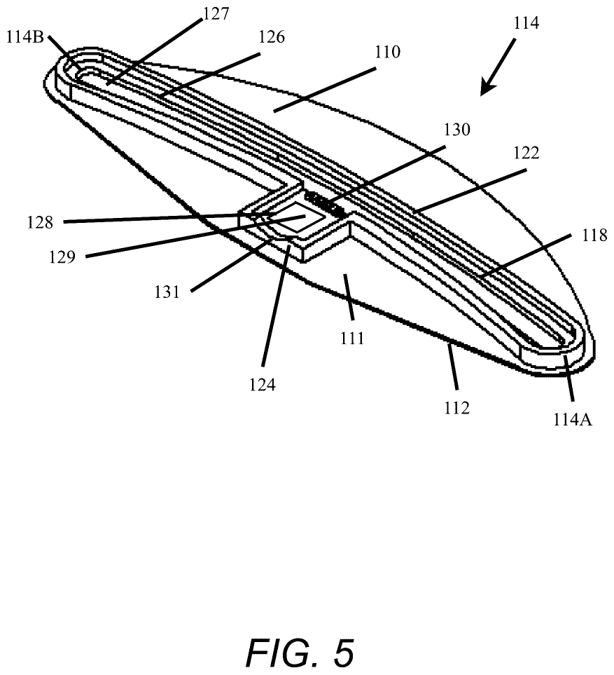

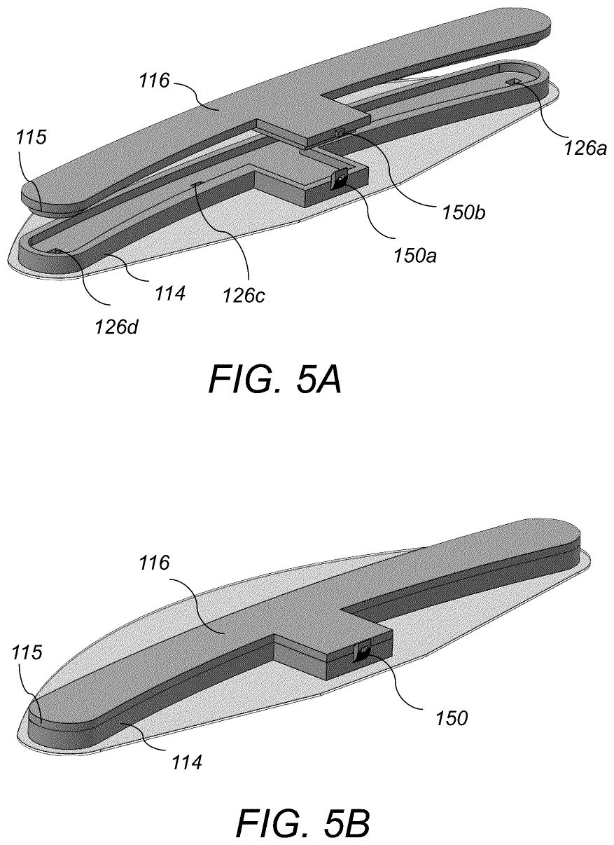

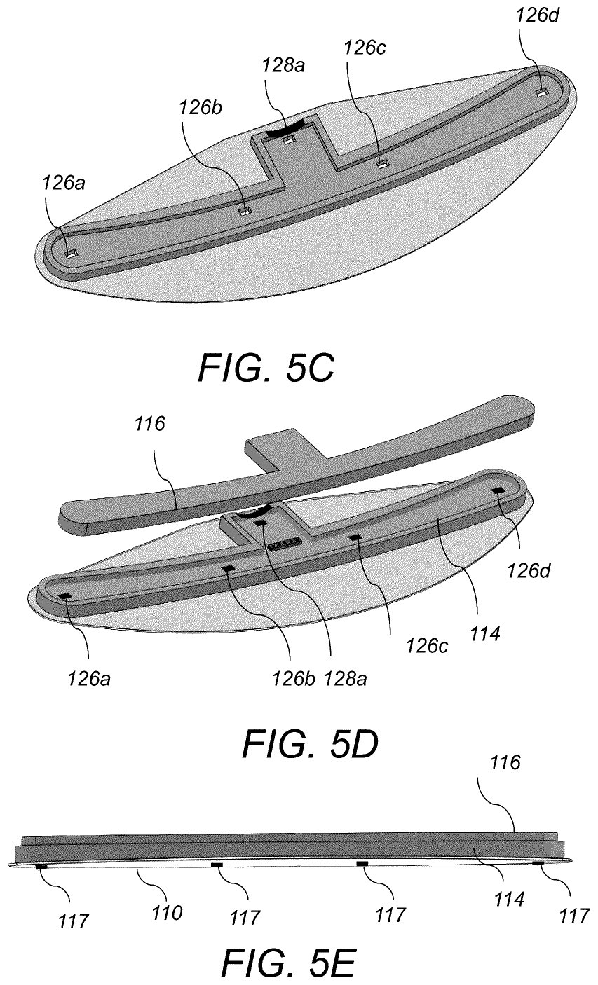

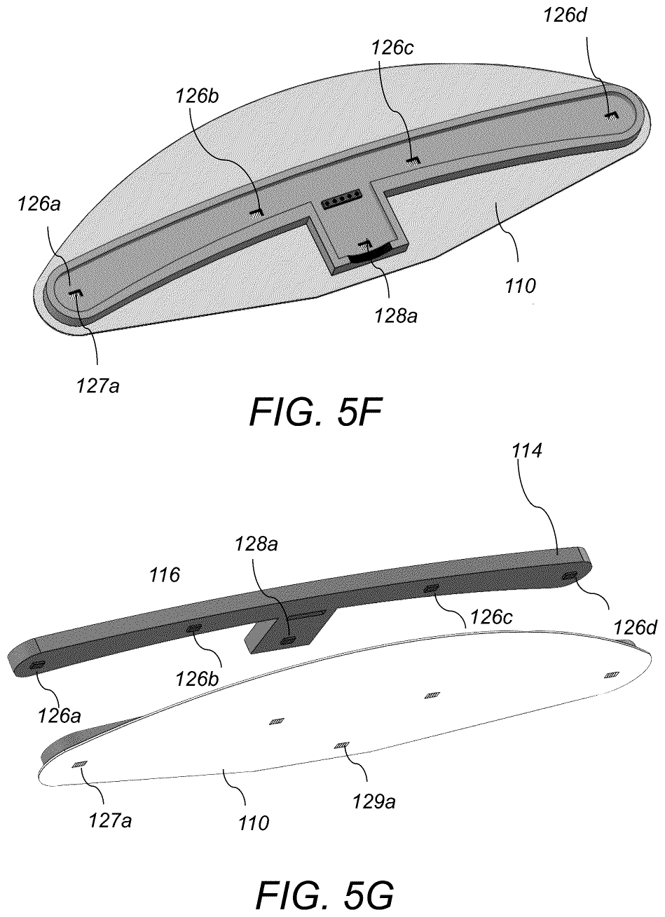

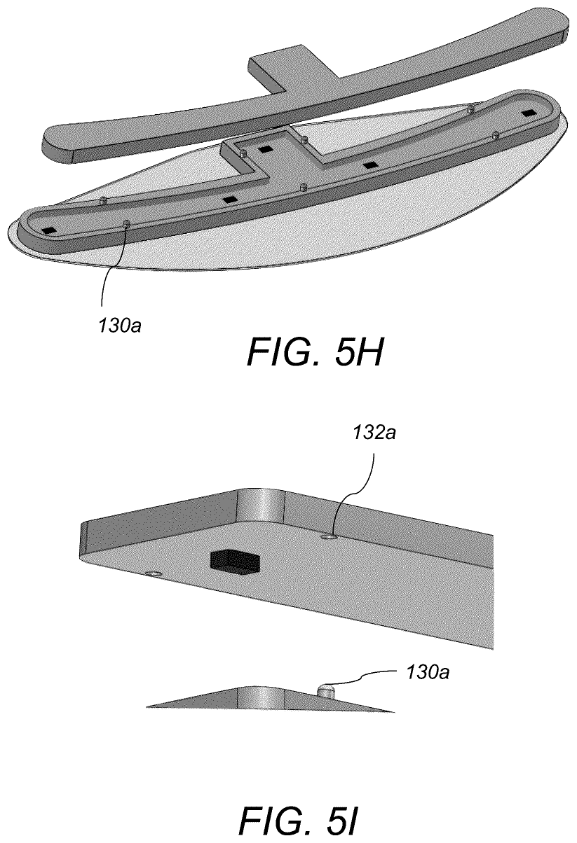

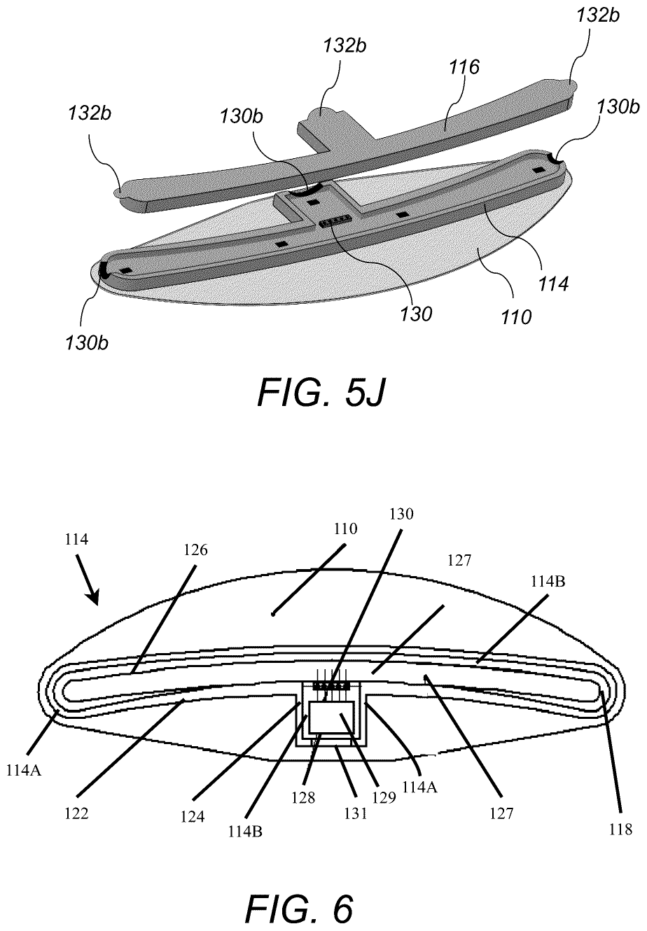

Referring to FIG. 5, the dock 114 is attached to the adhesive layer 110, and the cavity 118 is more clearly shown. The first dock portion 122 includes at least one first opening 126 formed by the step 114B and the second dock portion 124 includes at least one second opening 128 formed by the step 114B. Each of the first and second openings 126, 128 extends through the top and bottom surfaces 111, 112 of the adhesive layer 110, such that the first and second dock portions 122, 124 would be open to the skin of the user when the infection detection device 101 is attached to the skin of the user. Accordingly, when the infection detection device 101 is in the assembled configuration, as shown in FIG. 4, a portion of the first monitor section 116A of the drive 116 would be open to the periwound PW of the user through the first opening 126 and a portion of the second monitor section 116B of the drive 116 would be open to the healthy skin HS of the user through the second opening 128. In a further exemplary embodiment, instead of the dock 114 having the step 114B, the drive 116 may have an overhang 115 that extends over the top of and couples to the exterior wall 114A to couple the drive 116 to the dock 114 and prevent separation thereof, as shown in FIG. 5A and FIG. 5B. In an exemplary embodiment, the first dock section 122 includes a first conductive layer 127 extending through the first opening 126 and the second dock section 124 includes a second conductive layer 129 extending through the second opening 128. The first conductive layer 127 is configured to conduct heat dissipating from the periwound PW of the user, and the second conductive layer 129 is configured to conduct heat dissipating from the healthy skin HS of the user. The conductive layers 127 and 129 may be made of any heat conducting material, such as polyimide, Teflon™, adhesive film, epoxy, metal, among others. Accordingly, when the infection detection device 101 is in the assembled configuration, as shown in FIG. 4, a portion of the first monitor section 116A of the drive 116 would contact the first conductive layer 127 and a portion of the second monitor section 116B of the drive 116 would contact the second conductive layer 129. In an exemplary embodiment, the first conductive layer 127 contacts the periwound PW of the user and the second conductive layer 129 contact the healthy skin HS of the user.

In continued reference to FIG. 5, and reference to FIG. 5A-FIG. 5J, in a further embodiment, instead of having a larger singular opening (e.g., the first opening 126), the first dock portion 122 has a plurality of small openings 126a-126d extending through the top and bottom surfaces 111, 112 of the adhesive layer 110. In this embodiment, the cross-section areas of the small openings 126a-126d are smaller than a cross-section area of the first opening 126. Each of the small openings 126a-126d would be sized and shaped to receive a sensor 117 extending from the drive 116 therethrough. Similarly, instead of having a larger singular opening (e.g., the second opening 128), the second dock portion 124 has at least one smaller opening 128a extending through the top and bottom surfaces 111, 112 of the adhesive layer 110. The cross-section area of the smaller opening 128a is smaller than a cross-section area of the second opening 128. The at least one smaller opening 128a would be sized and shaped to receive a sensor 117 extending from the drive 116 therethrough. In a yet further exemplary embodiment, each of the smaller openings 126a-126d of the first dock portion and each of the at least one smaller opening 128a of the second dock portion 124 have a conductive layer 127a, 129a similar to the first and second conductive layers 127, 129 for conducting heat from at least one of the periwound PW of the user and the healthy skin HS of the user, respectively, as shown in FIG. 5F and FIG. 5G.

In continued reference to FIG. 5, the dock 114 includes the coupling mechanism 130 for coupling the drive 116 to the dock 114 when the drive 116 is inserted into the cavity 118 of the dock 114 as shown in FIG. 4. In an embodiment, the coupling mechanism 130 extends outward from the dock 114 and is sized and shaped to be inserted into the drive coupling mechanism 132 of the drive 116, as shown in FIG. 8. Once received by the drive coupling mechanism 132, the dock coupling mechanism 130 couples to the drive coupling mechanism 132. In an embodiment, the dock coupling mechanism 130 may have a plurality of holes extending vertically therethrough, each of the holes sized and shaped to receive one of a plurality of pegs extending vertically through the drive coupling mechanism 132. In another embodiment, the drive coupling mechanism 132 includes a plurality of openings 132a and each of the openings is sized and shaped to receive one of a plurality of pegs 130a extending vertically from the dock 114, as shown in FIG. 5H and FIG. 5I. In yet another example, the drive coupling mechanism 132 may have at least one tab 132b extending therefrom, the at least one tab 132b being sized and shaped to be inserted into a correspondingly sized and shaped window 130b of the dock coupling mechanism 130, as shown in FIG. 5J. In a further embodiment, the dock coupling mechanism 130 may include a locking mechanism 150 for locking the drive 116 in the cavity 118 relative to the dock 114, as shown in FIG. 5A and FIG. 5B. The locking mechanism 150 includes a dock locking element 150a that is shaped and sized to receive a drive locking element 150b. In one example, the dock locking element 150a is a tab that includes an opening sized and shaped to frictionally receive a protrusion of the drive locking element 150b, as shown in FIG. 5A and FIG. 5B.

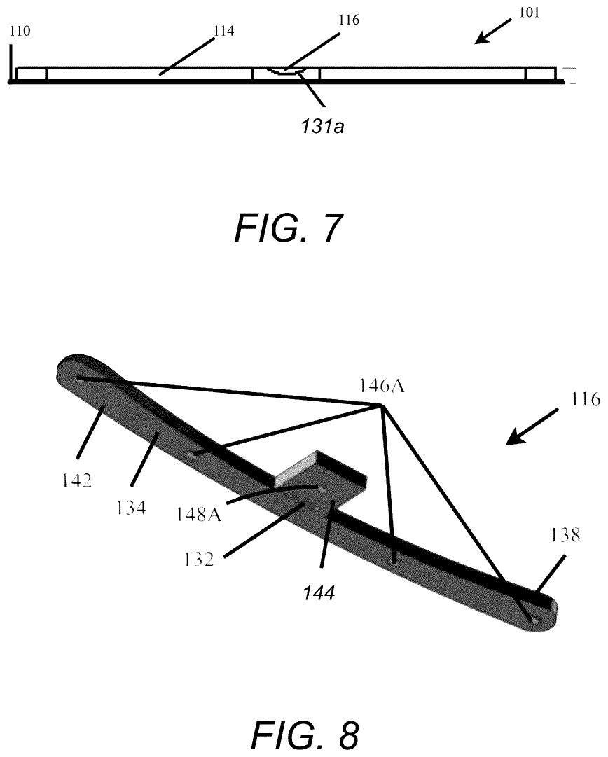

In continued reference to FIG. 5, the dock 114 may further include a separation mechanism 131 for separating the drive 116 from the dock 114. The separation mechanism 131 may be one of an indentation, a tab, a lever, or a press-fit mechanism, among others. In an exemplary embodiment, the separation mechanism may include an indentation 131a along the exterior wall 114A at the second dock section 124, as shown in FIG. 7. The indention 131a is sized to be approximately similar to a fingertip of the user, such that the user may insert their fingertip into the indentation 131a and remove the drive 116 from the dock 114. Although the exemplary embodiment shows a singular indentation 131a, the dock 114 may have multiple indentations along any portion(s) of the exterior wall 114A such that the user may uncouple the drive coupling mechanism 132 from the dock coupling mechanism 130 and separate the drive 116 from the dock 114. In a further embodiment, instead of an indentation, the separation mechanism may include a tag 132b configured to be receded to reveal an indentation 130b, shown in FIG. 5J. In a yet further embodiment, the separation mechanism may include a lever configured to be actuated by the user to lift the drive 116 out of the cavity 118 the dock 114.

Referring to FIG. 6 the dock 114 includes the first dock section 122, the second dock section 124, and the dock coupling mechanism 130. The first and second dock sections 122, 124 include the first and second openings 126, 128, respectively, and the first and second conductive layers 127, 129, respectively. The dock 114 further includes the cavity 118, the indentation 131a, the exterior wall 114A and the step 114B. In an embodiment, the first dock section 122 and the first monitor section 116A of the drive 116 (shown in FIG. 3) are sized and shaped to conform to a shape of the caesarean incision CI (shown in FIG. 1). In further embodiments, the first dock section 122 and the first monitor section 116A of the drive 116 may be any shape to conform to any wound or incision. For example, the first dock portion 122 and the first monitor section 116A may conform to a shape of an appendectomy incision or a heart bypass incision. Further, the second dock section 124 and the second monitor section 116B of the drive 116 (shown in FIG. 3) may be sized and shaped to be wider and to extend further from the first dock section 122 and the first monitor section 116A, respectively.

Referring to FIG. 7, when the infection detection device 101 is in the assembled configuration, the drive 116 is inserted into the cavity 118 (shown in FIG. 3) of the dock 114 such that a top surface of the drive 116 is flush with a top surface of the dock 114. In an embodiment, the infection detection device 101 is very thin and prevents the infection detection device 101 from interfering with the bandage 120 (shown in FIG. 1) or any other clothing or fabric that may be covering the infection detection device 101. In one example, in the assembled configuration, the infection detection device 101 is approximately 4 inches in length, 0.4 inches in width and 0.10 inches in height. In an embodiment, the dimensions of an infection detection device are dependent on the shape of the infection detection device and the wound that the infection detection device is configured to monitor. Additionally, the infection detection device 101 may be configured to have a contour (e.g., a bend) to conform to a shape of the wound or incision and/or to an anatomical contour of the body of the user in an area of the wound or incision. In an embodiment, the infection detection device 101 is made from flexible material and may be bent and shaped to conform to the surface of any body part. In this way, the device 101 is operable to detect wound infections at any location that is needed.

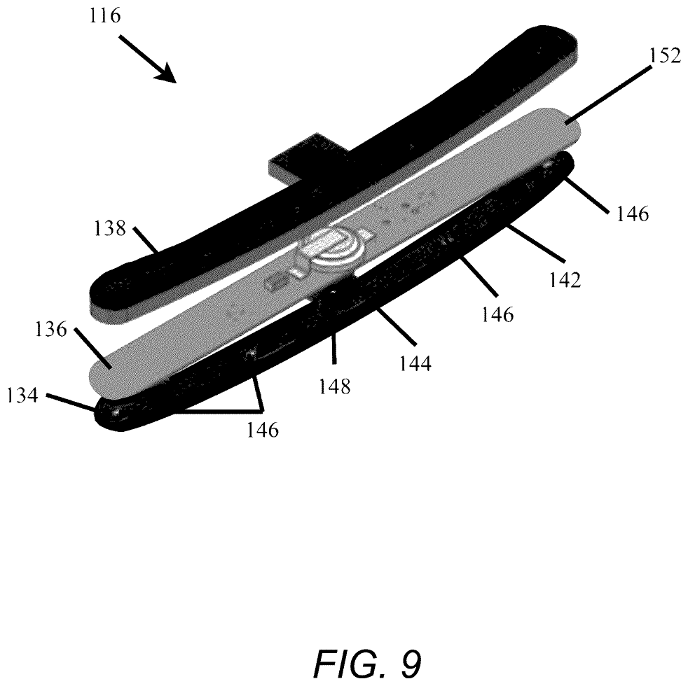

Referring to FIG. 8 and FIG. 9, the drive 116 includes a base 134, a printed circuit board (PCB) 136, and a cover 138. The base 134 and cover 138 are sized and shaped to be snuggly inserted into the cavity 118 of the dock 114 (shown in FIG. 6). The base 134 includes a first base portion 142 and a second base portion 144. In an embodiment, the first base portion 142 includes a plurality of first base openings 146, each of the first base openings 146 being configured to receive therein a first sensor 146A (shown in FIG. 8) extending from the PCB 136. Similarly, the second base portion 144 includes at least one second base opening 148, and the at least one second base opening 148 is configured to receive therein a second sensor 148A (shown in FIG. 8) extending from the PCB 136. In a further embodiment, instead of the PCB 136, the drive 116 may include a plurality of wires and leads connecting the temperature sensors 146A, 148A to a microcontroller or a processor.

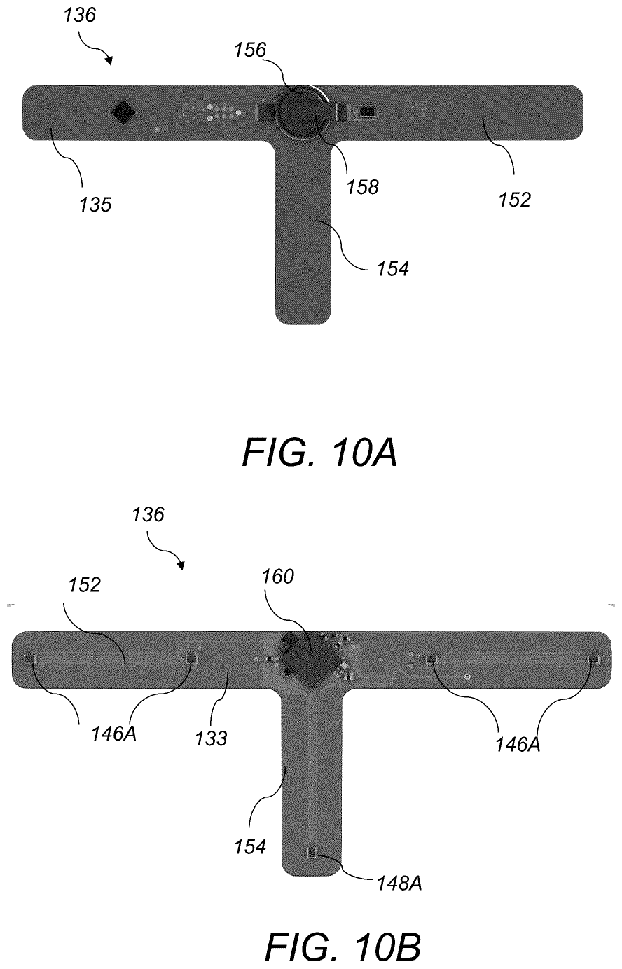

Referring to FIG. 8-FIG. 10B, the PCB 136 is sized and shaped to correspond to the size and shape of the base 134. In an exemplary embodiment, the PCB 136 is substantially rigid, however, in a further exemplary embodiment, the PCB 136 is substantially flexible. In an exemplary embodiment, the PCB 136 includes a first PCB section 152 and a second PCB section 154 (shown in FIG. 10A). As mentioned above, the PCB 136 further includes a plurality of first sensors 146A (e.g., first measurement temperature sensors 146A) along the first PCB section 152 (shown in FIG. 10B) and at least one second sensor 148A (e.g., second reference temperature sensor 148A) along the second PCB section 154 (shown in FIG. 10B). In an embodiment, the sensors 146A, 148A are digital temperature sensors that are configured to continuously and consistently measure the temperatures from the periwound PW (shown in FIG. 1) and the healthy skin HS (shown in FIG. 1) respectively. For example, the temperature sensors 146A, 148A may measure the temperatures every predetermined amount of time (e.g., every second, every minute, every hour, every day, etc.). In an embodiment, the temperature sensors 146A, 148A may be thermistors, analog temperature sensors, or other types of sensors known in the art.

In continued reference to FIG. 8-FIG. 10B, the first temperature sensors 146A (shown in FIG. 10B) are configured to measure a temperature of the periwound PW (shown in FIG. 1) adjacent to the caesarean incision CI (shown in FIG. 1). Each of the first temperature sensors 146A extends outward from a bottom surface 133 (shown in FIG. 10B) of the PCB 136, specifically from the first PCB section 152, and through a respective one of the first base openings 146 of the first base portion 142 to contact the first conductive layer 127 (shown in FIG. 6) of the first dock section 122. The first conductive layer 127 conducts heat from the periwound PW to the first temperature sensors 146A. Measurement data is then collected from the first temperature sensors 146A and transmitted to the user device 102 (shown in FIG. 1) via the network 103 (shown in FIG. 1), as will be described in detail below. In a further embodiment, instead of contacting the first conductive layer 127, the first temperature sensors 146A may directly contact the periwound PW through the first opening 126, or through the plurality of smaller openings 126a as described above. Additionally, although FIG. 10B shows the PCB 136 having four first temperature sensors 146A, in a further embodiment, the PCB 136 may have any number of the first temperature sensors 146A configured to measure the temperature of the periwound PW of a user.

In continued reference to FIG. 8-FIG. 10B, the second temperature sensor 148A (shown in FIG. 10B) is configured to measure a temperature of the healthy skin HS (shown in FIG. 1) adjacent to the periwound PW (shown in FIG. 1). The second temperature sensor 148A extends outward from the second PCB section 154, and through the second base opening 148 of the second base portion 144 to contact the second conductive layer 129 of the second dock section 124 (shown in FIG. 6). The second conductive layer 129 conducts heat from the healthy skin HS to the second temperature sensor 148A. Reference data is then collected from the second temperature sensor 148A and transmitted to the user device 102 (shown in FIG. 1) via the network 103 (shown in FIG. 1). In a further embodiment, the second temperature sensor 148A may directly contact the healthy skin HS through the second opening 128, or through the smaller opening 128a as described above. Additionally, although FIG. 10B shows the PCB 136 having one second temperature sensor 148A, in a further embodiment, the PCB 136 may have any number of the second temperature sensors 148A configured to measure the temperature of a portion of healthy skin HS adjacent to a periwound PW of a user.

In continued reference to FIG. 8, drive 116 includes the drive coupling mechanism 132 that is configured to receive the dock coupling mechanism 130 (shown in FIG. 6) to couple the drive 116 to the dock 114. In the exemplary embodiment, the drive coupling mechanism 132 extends partially through the drive 116. Specifically, the drive coupling mechanism 132 extends through the base 134, the PCB 136 and into the cover 138. In a further embodiment, the receiving element 132 may be along a perimeter of the cover 138 and is configured to couple to the coupling mechanism 130 along a perimeter of the dock 114, as was shown in FIG. 5H and FIG. 5I. In an embodiment, the dock 114 and the drive 116 are composed of biocompatible materials, such as silicone, polypropylene, or polyethylene, among others.

In continued reference to FIG. 9, the cover 138 of the drive 116 is configured to be coupled to the base 134 with the PCB 136 therebetween. The cover 138 may be coupled to the base 134 via any method know to one with ordinary skill in the art, such that the cover 138 is aligned with the base 134 and PCB 136. In a further exemplary embodiment, the cover 138 may be integrally formed with the base 134 such that the PCB 136 may not be removed therefrom.

Referring to FIG. 10A and FIG. 10B, the PCB 136 includes a power supply 156 (e.g., a battery 156) held in place by a support 158 and a microcontroller 160. The battery 156 is coupled to the PCB 136 and provides power to the microcontroller 160, the first temperature sensors 146A and the second temperature sensor 148A. The support 158 couples the battery 156 to the PCB 136. The support 158 may be any support known to one with ordinary skill in the art. For example, the support 158 may extend across a top portion of the battery 156, similar to a cover, to couple the battery 156 to the PCB 136. In another example, the support 158 may extend outward from a top surface 135 of the PCB 136 and around a bottom portion of the battery 156, such that the battery 156 is inserted into the support 158. In an embodiment, the battery 156 may be removed and replaced with a new battery 156.

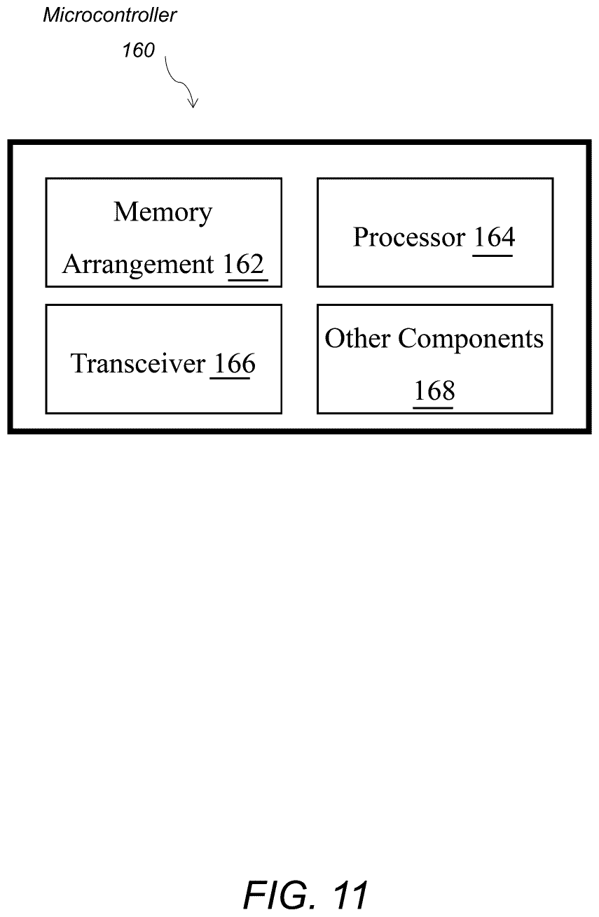

Referring to FIG. 11, microcontroller 160 includes a memory arrangement 162, a processor 164, a transceiver 166, and other components 168. The memory arrangement 162 of this embodiment is a hardware component configured to store data related to operation of the drive 116. For example, the memory arrangement 162 may be configured to store the periwound PW data collected by the first temperature sensors 146A and the healthy skin HS data collected by the second temperature sensor 148A (i.e., collectively the collected data from the temperature sensors 146A, 148A), as well as data output by the processor 164.

In continued reference to FIG. 11, in the exemplary embodiment, the processor 164 may execute a program that interprets the collected data from the temperature sensors 146A, 148A (shown in FIG. 10B) to make a determination about whether the wound/caesarean incision CI (shown in FIG. 1) is infected. Furthermore, the transceiver 166 in this embodiment is a hardware component configured to establish a connection with the network 103 (shown in FIG. 1) and the user device 102 (shown in FIG. 1) to transmit the collected data from the temperature sensors 146A, 148A and/or the data output by the processor 164 to the user device 102. Accordingly, the transceiver 166 may operate on a variety of different frequencies as appropriate. For example, a chosen frequency of the transceiver 166 will operate on a chosen frequency of the transceiver 106 of the user device 102. Although the battery 156, the support 158, and the microcontroller 160 are shown in FIG. 8-FIG. 10B as coupled to the first PCB portion 152, in a further embodiment, the battery 156, the support 158, and the microcontroller 160 may be coupled to the second PCB portion 154, or divided amongst the first PCB portion 152 and the second PCB portion 154, as long as the battery 156, the support 158, and the microcontroller 160 are coupled to the PCB 136.

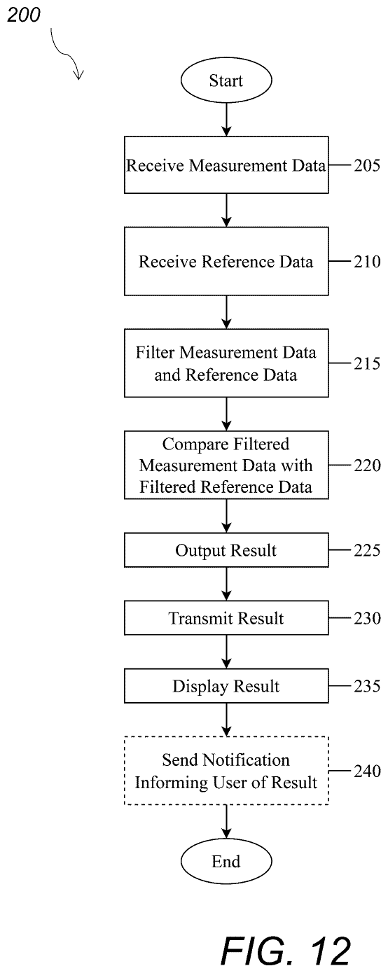

Referring to FIG. 12, a process of operation 200 may be taken by the drive 116 and its microcontroller 160 (shown in FIG. 11) that determines whether a wound, such as the shown caesarean incision CI, is infected. Process 200 includes the following steps 205-240. In step 205, the processor 164 receives a set of wound data (i.e., measurement data) from the first temperature sensors 146A. The measurement data may refer to temperatures measured at different points along the periwound PW. In step 210, the processor 164 receives the reference data from the second temperature sensor 148A. The reference data may refer to a temperature measured at the healthy skin HS. It should be noted that while steps 205 and 210 are listed in sequential order, they may be performed in any order as appropriate, including contemporaneously with each other.

In step 215, the processor 164 filters the measurement data and the reference data to remove from calculations any data that is more than likely to be incorrect. When reviewing the measurement data, the processor 164 may ignore a temperature that is substantially higher or substantially lower than the rest of the temperatures. For example, if one of the first temperature sensors 146A returns a temperature of 34° C. and the remaining first temperature sensors 146A return temperatures ranging from 30° C. to 32° C., the processor 164 may ignore the 34° C. temperature from the first one of the first temperature sensors 146A. The purpose of step 215 is to remove any data that is more than likely incorrect and would skew the final output of the method 200. Additionally, if one of the first temperature sensors 146A consistently returns temperatures that are more than likely incorrect, the user will be notified to review the faulty first temperature sensor 146A, the faulty first temperature sensor 146A may be shut off, or data from the faulty first temperature sensor 146A will be ignored.

In step 220, the processor 164 compares the filtered measurement data to the filtered reference data to determine whether the wound/caesarean incision CI is infected. For example, the processor 164 may average the filtered measurement data, average the filtered reference data, and compares the averages, such that, if the difference between the averages is greater than a predetermined threshold, then the wound/caesarean incision CI may be infected. If the difference between the averages is less than the predetermined threshold, then the wound/caesarean incision CI is unlikely to be infected. In step 225, the processor 164 outputs the determination of whether the wound/caesarean incision CI is infected. In step 230, the output is transmitted by the transceiver 166 of the microcontroller 160 and is received by the transceiver 106 of the user device 102. In step 235, the output is displayed on the display device 108 of the user device 102. In a further, optional, step 240, the user device 102 may send out a notification (e.g., a sound notification, a visible notification) to the user informing the user that the wound/caesarean incision CI is infected and that the user should see a doctor or physician.

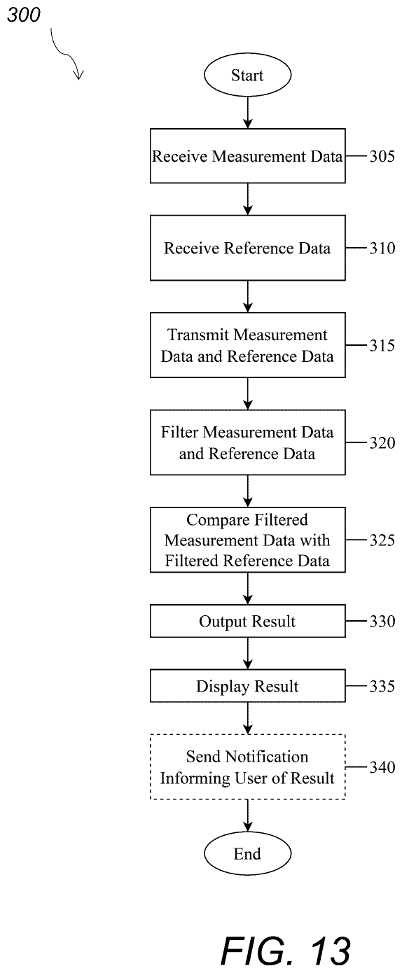

Referring to FIG. 13, an exemplary method 300 for determining whether the wound/caesarean incision CI is infected includes the following steps 305-340. The method 300 may be run via an algorithm stored on the user device 102 instead of on the microcontroller 160. In step 305, the processor 164 receives wound data (i.e., measurement data) from the first temperature sensors 146A. The measurement data refers to temperatures measured at different points along the periwound PW. In step 310, the processor 164 receives the reference data from the second temperature sensor 148A. The reference data refers to a temperature measured at the healthy skin HS. In step 315, the measurement data and the reference data are transmitted by the transceiver 166 of the microcontroller 160 to the transceiver 106 of the user device 102. It should be noted that while steps 305 and 310 are listed in sequential order, they may be performed in any order as appropriate, including contemporaneously with each other.

In step 320, the processor 104 filters the measurement data and the reference data in a substantially similar manner as the processor 164 filters the measurement data and the reference data in method 200. In step 325, the processor 104 compares the filtered measurement data to the filtered reference data to determine whether the wound/caesarean incision CI is infected in a substantially similar manner as the processor 164 compares the filtered measurement data and the filtered reference data in method 200. In step 330, the processor 104 outputs the determination of whether the wound/caesarean incision CI is infected. In step 335, the output is displayed on the display device 108 of the user device 102. In a further, optional, step 340, the user device 102 may send out a notification (e.g., a sound notification, a visible notification) to the user informing the user that the caesarean incision CI is infected and that the user should see a doctor or physician.

Other embodiments include one or more of the following. The user may capture photos of the periwound PW and/or the infection detection device 101 on the periwound PW and upload them to the user device 102 and/or an application on the user device 102. The user may capture photos of the periwound PW and/or the infection detection device 101 on the periwound PW using the user device 102 and directly upload the photos onto an application on the user device 102. The photos may be processed and analyzed via software contained on the application to improve infection detection accuracy, troubleshoot configuration of the infection detection device 101, and for other purposes that would be known to those skilled in the art. Additional inputs may be entered, captured and/or transmitted to the user device 102 and/or an application on the user device 102. The additional inputs include pain scale ratings, body temperature, ambient temperature, chills and sweat data, redness, soreness and swelling data, contact bleeding data, wound discharge data, wound odor data, vomiting, wound healing progress data, wound tissue granulation data, wound culture data, blood pressure data, heart rate data, oximeter data, level of consciousness, full blood count data, white blood cell count data, C-reactive protein data, plasma viscosity, erythrocyte sedimentation rate, microbial DNA data, bacterial protease activity, among others.

Several embodiments of the present invention have been described. Nevertheless, it will be understood that various modifications may be made without departing from the spirit and scope of the invention. Accordingly, other embodiments are within the scope of the following claims.

What is claimed is:

1. An infection detection device configured to continuously monitor a temperature of a periwound of a user, comprising:
a dock including an adhesive layer having an adhesive coating on a wound side thereof, the dock having a first dock portion and a second dock portion; and
a drive configured to be inserted into a cavity of the dock, the drive including:
a printed circuit board positioned along a base of the drive, the printed circuit board having a first printed circuit board portion and a second printed circuit board portion;
a plurality of first temperature sensors extending from the first printed circuit board portion and through the first dock portion; and
at least one second temperature sensor extending from the second printed circuit board portion and through the second dock portion.

2. The device of claim 1, wherein the dock further comprises a coupling mechanism configured to couple the drive to the dock.

3. The device of claim 2, wherein the coupling mechanism is configured to be inserted into a receiving element of the drive to couple the drive to the dock.

4. The device of claim 1, wherein the first dock portion includes a first opening extending through the adhesive layer to the periwound and the second dock portion includes a second opening extending through the adhesive layer to a portion of skin adjacent to the periwound.

5. The device of claim 4, wherein the first dock portion includes a first conductive layer spanning the first opening, the first conductive layer contacting the periwound and the first temperature sensors, and wherein the second dock portion includes a second conductive layer spanning the second opening, the second conductive layer contacting the portion of skin adjacent to the periwound and the at least one second temperature sensor.

6. The device of claim 1, wherein the first dock portion includes a plurality of openings, each of the plurality of openings sized and shaped to receive a corresponding one of the first temperature sensors therethrough.

7. The device of claim 6, wherein each of the plurality of openings including a conductive layer contacting the periwound and the corresponding one of the first temperature sensors.

8. The device of claim 1, wherein the dock is contoured to correspond to a shape of an incision.

9. The device of claim 1, wherein the drive is configured to be inserted into the cavity of the dock such that a top surface of the drive is flush with a top surface of the dock.

10. An infection detection system configured to continuously monitor a temperature of a periwound, comprising:
an infection detection device including:
a dock coupled to an adhesive layer having an adhesive coating on a wound side thereof, the dock having a measurement portion and a reference portion; and
a drive configured to be inserted into a cavity of the dock, the drive including:
a printed circuit board positioned along a base of the drive, the printed circuit board having a first printed circuit board portion and a second printed circuit board portion;
a plurality of first temperature sensors extending from the first printed circuit board portion and through the first dock portion; and
at least one second temperature sensor extending from the second printed circuit board portion and through the second dock portion; and
a microcontroller configured to compare measurement data from the plurality of first temperature sensors and reference data from the at least one second temperature sensor,
wherein the infection detection device is configured to communicate with a remote communication device.

11. The system of claim 10, wherein the microcontroller is configured to transmit the comparison of the measurement data and the reference data and an output of the microcontroller to the remote communication device.

12. The system of claim 10, wherein the microcontroller is configured to transmit the measurement data and the reference data to the remote communication device.

13. The system of claim 10, wherein the dock further comprises a coupling mechanism configured to couple the drive to the dock.

14. The system of claim 10, wherein the first dock portion includes a first opening extending through the adhesive layer to the periwound and the second dock portion includes a second opening extending through the adhesive layer to a portion of skin adjacent to the periwound.

15. The system of claim 14, wherein the first dock portion includes a first conductive layer spanning the first opening, the first conductive layer contacting the periwound and the first temperature sensors, and wherein the second dock portion includes a second conductive layer spanning the second opening, the second conductive layer contacting the portion of skin adjacent to the periwound and the at least one second temperature sensor.

16. The system of claim 10, wherein the first dock portion includes a plurality of openings, each of the plurality of openings sized and shaped to receive a corresponding one of the first temperature sensors therethrough.

17. The system of claim 16, wherein each of the plurality of openings including a conductive layer contacting the periwound and the corresponding one of the first temperature sensors.

18. The system of claim 10, further comprising a bandage configured to cover the infection detection device and the periwound.

19. A method for determining occurrence of a wound infection, comprising:
collecting measurement data by measuring temperatures of periwound surrounding the wound via a plurality of first temperature sensors;
collecting reference data by measuring a temperature of a portion of skin adjacent to the periwound via at least one second temperature sensor;
filtering erroneous temperatures from the measurement data and the reference data;
comparing the filtered measurement data to the filtered reference data;
outputting an indication of a wound infection based on the comparison of the filtered measurement data to the filtered reference data; and
wherein the plurality of first temperature sensors extend from a first portion of a printed circuit board of a drive, the at least one second temperature sensor extends from a second portion of the printed circuit board of the drive, and the drive is configured to be inserted into a cavity of a dock couplable to at least one of the periwound and the portion of skin adjacent to the periwound via an adhesive layer.

20. The method of claim 19, further comprising displaying the output on a remote communication device.

21. The method of claim 19 further comprising collecting and entering additional measurement data, wherein the additional measurement data comprise one or more of pain scale ratings, body temperature, ambient temperature, chills and sweat data, redness, soreness and swelling data, contact bleeding data, wound discharge data, wound odor data, vomiting, wound healing progress data, wound tissue granulation data, wound culture data, blood pressure data, heart rate data, oximeter data, level of consciousness, full blood count data, white blood cell count data, C-reactive protein data, plasma viscosity, erythrocyte sedimentation rate, microbial DNA data, and bacterial protease activity.
